(19) United States
(12) Patent Application Publication
(10) Pub. No.: US 2005/0010181 A1
(43) Pub. Date: Jan. 13, 2005

(54) External male catheter having weighted pickup

(76) Inventor: Donald Dolan, Kouts, IN (US)

Correspondence Address: Shoemaker and Mattare, Ltd., Suite 100, 10 Post Office Road, Silver Spring, MD, 20910, US

(21) Appl. No.: 10/912,086

(22) Filed: Aug. 6, 2004

Related U.S. Application Data
(63) Continuation-in-part of application No. 10/375,065, filed on Feb. 28, 2003.
(60) Provisional application No. 60/359,905, filed on Feb. 28, 2002.

(51) Int. Cl. A61F 5/44; A61M 27/00

(52) U.S. Cl. 604/351; 604/544

(57) ABSTRACT

An external urinary catheter includes a hollow body which is placed over the penis to receive urine passing from the urethra. In one embodiment of the invention, the body has a front surface which is hemispherical and a rear surface which is hemitoroidal. Vacuum tubing, passing through the wall of the body leads to a weighted pickup inside the container, extracts urine from the body and delivers it to a remote receptacle. The weighted pickup remains at the lowest point of the body, no matter what the wearer's orientation.

This application is a continuation-in-part of Ser. No. 10/375065, filed Feb. 28, 2003, which claimed benefit of provisional application No. 60/359905, filed Feb. 28, 2002.

### BACKGROUND OF THE INVENTION

This invention relates to the field of medicine, and more particularly to external urinary catheters for men.

Loss of continence may be embarrassing and is inconvenient. Prior inventors have proposed a variety of solutions to this problem. To collect urine from people who have diminished or no bladder control, a variety of absorbent pads and catheters have been developed. Pads are uncomfortable when wet, and may leak. Internal catheters also cause discomfort and may reduce the patient's mobility.

Some external urine collection devices have been designed for men. Typically, with such devices, the penis is placed in a urine receptacle leading to a remote disposal container. We refer to such devices as “external urinary catheters”.

This invention provides an improved solution to the incontinence problem for men.

### BRIEF DESCRIPTION OF THE DRAWINGS

In the accompanying drawings,

FIG. 1 is an exploded perspective view of an external male catheter embodying the invention;

FIG. 2 is a sectional view of the device, assembled, taken on a bisecting longitudinal plane;

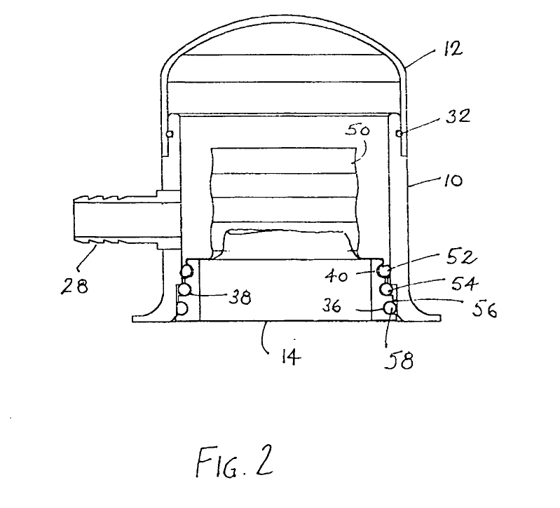

FIG. 3 is a view like FIG. 1 of a second embodiment of the invention;

FIG. 4 is a sectional view of the device, assembled, taken on a bisecting longitudinal plane;

FIG. 5 is a view like FIG. 1 of a third embodiment of the invention;

FIG. 6 is a sectional view of the device, assembled, taken on a bisecting longitudinal plane;

FIG. 7 is a sectional side elevation of a fourth embodiment of the invention; and

FIG. 8 is a sectional end view of the device shown in FIG. 7.

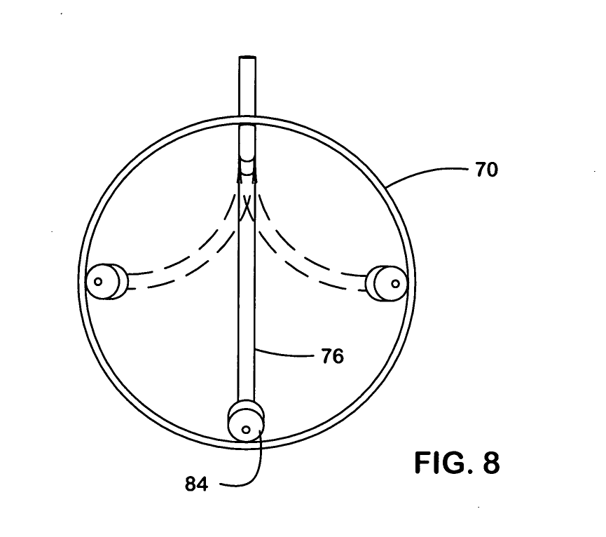

### DESCRIPTION OF THE PREFERRED EMBODIMENT

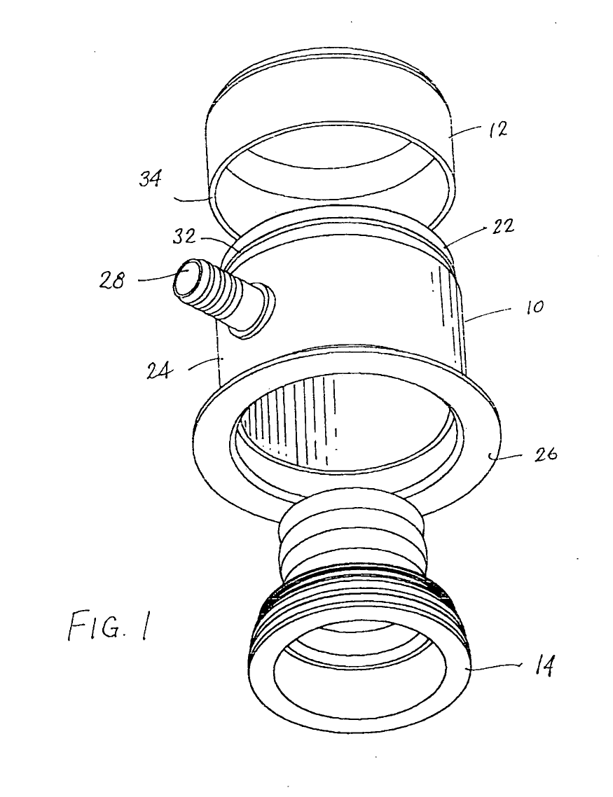

As shown in FIG. 1, an external urinary catheter embodying the invention includes a housing 10, a removable base 12, and a cap 14. These parts are preferably machined from ultra-high molecular weight polyethylene (UHMW); however, other materials, and other methods of manufacture, such as injection molding, may prove satisfactory.

The housing 10 has a substantially uniform inner diameter sized to fit over the penis. Its outer surface has a reduced-diameter upper rim 22, a main portion 24, and a lower circumferential flange or brim 26. A hollow tubing fitting 28 is installed in a hole extending through the wall of the main portion. The fitting directs liquid which collects within the device to tubing (not shown) which leads to a bag or other receptacle at a lower elevation. The bag may be worn by the patient, for example, on the leg. For stationary patients, the bag may be attached to a suitable fixture.

A groove for an upper O-ring 32 is formed in the surface of the upper rim.

The cap 14 is a simple piece of uniform thickness, having a cylindrical skirt 34 whose inner diameter is a slip fit over the upper rim of the housing. The upper O-ring 32 presses outwardly against the skirt when the cap is installed, retaining the cap in place and providing a fluid seal.

The base 12 is a ring-shaped element having three external grooves 36, 38, 40. The upper groove 40 is designed to receive the base of a fist elastic, flaccid sleeve 50 having a relaxed diameter less than that of the penis, and substantially less than the inside diameter of the base. Removing the distal end from a condom produces a particularly suitable sleeve. The rolled proximal end 52 of the condom is stretched over the shoulder. An additional retainer (not shown) may be provided if desired. A second elastic, flexible sleeve 54 is seated in the middle groove 38. This sleeve is shorter than the first, and is designed to seal around the base of the penis, whereas the first sleeve is designed to seal near the head of the penis. The dual-sleeve arrangement provides particularly reliable leak protection.

An O-ring 58 is seated in the bottom groove 36. It is sized to provide a slight interference fit against the counterbore 56 at the bottom of the housing, to keep the parts assembled.

When the base (with the flaccid sleeve installed) is inserted from below into the housing, the lower O-ring provides a fluid seal against the housing, and also frictionally engages the inner diameter of the housing to hold the parts together. With the cap also in place, the assembly is placed over the penis, over which the sleeve stretches, forming a liquid-tight seal against it. The O-rings prevent leakage between the elements 10, 12 and 14, so that the fitting 28 is the only avenue of escape for urine which enters the housing through the urethra.

The device may be held in place simply by briefs worn over it. Additional retainers may be added as necessary. If desired, an absorbent pad or piece of material may be placed beneath the rim of the device, against the skin, to intercept any leakage when the device is removed, and to prevent chafing against the scrotum.

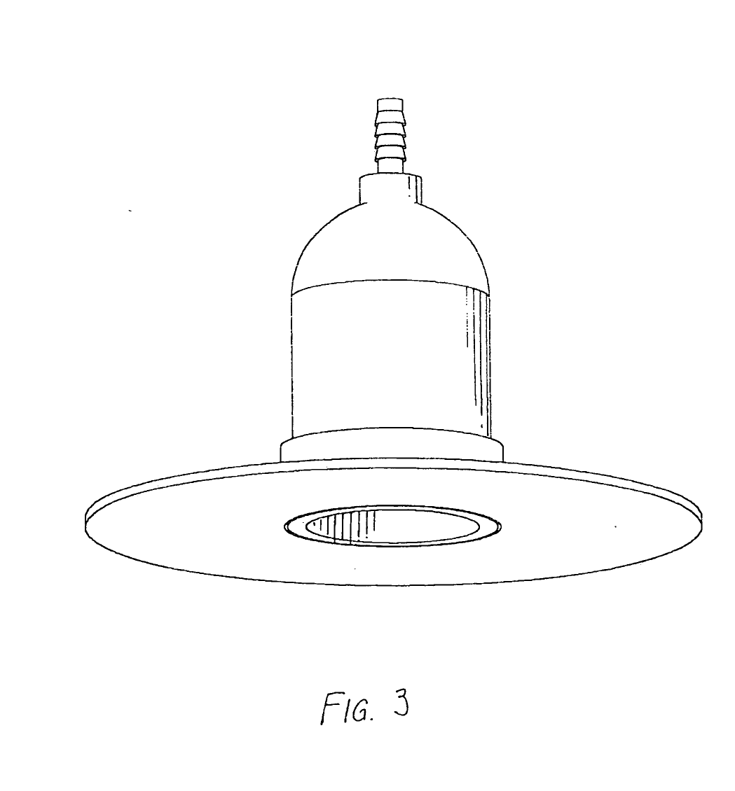

FIGS. 3-6 show two alternative forms of the invention, which in principle are the same.

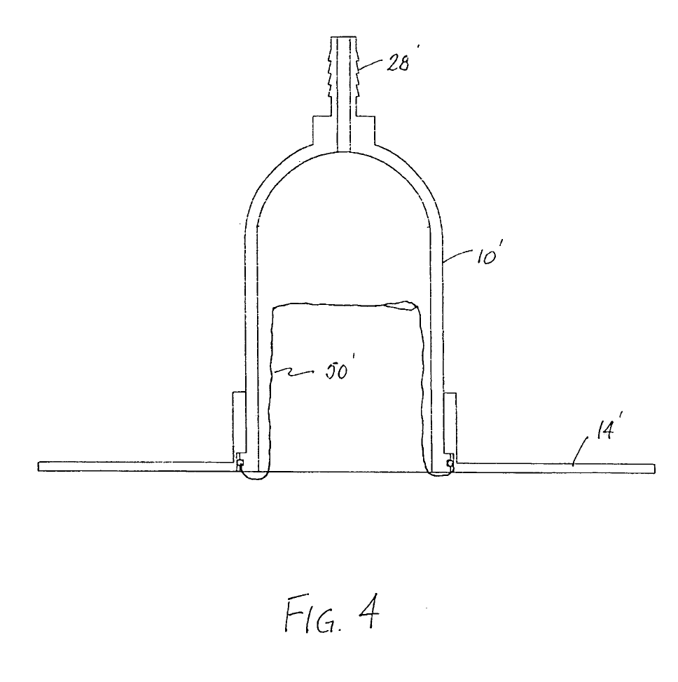

The device shown FIGS. 3 and 4 has a longer housing 10′, terminating at a fitting 28′ which extends along the longitudinal axis of the device. The rolled end of an elastic sleeve 50′ may be installed in the external groove at the base 14′ of the housing. Preferably, the sleeve is passed down under the bottom end of the housing, then up into it. The base is then pressed onto the housing from above, securing the rolled end of the sleeve in the groove. Friction maintains the assembly.

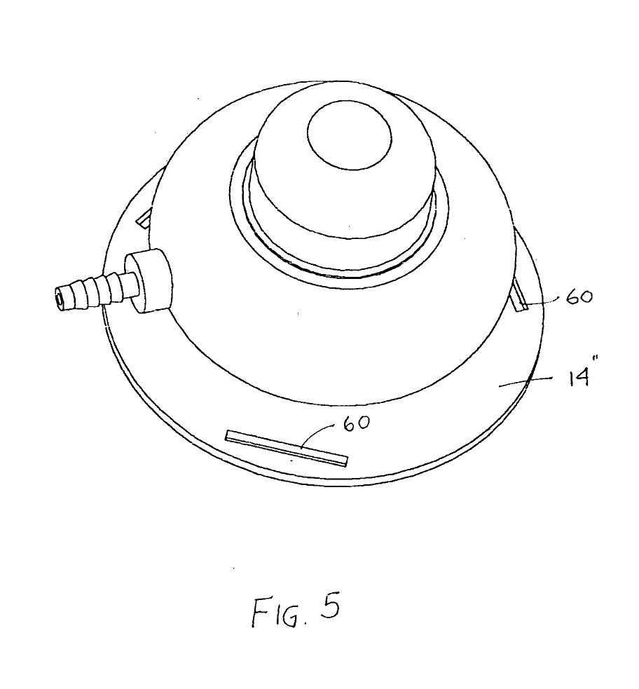

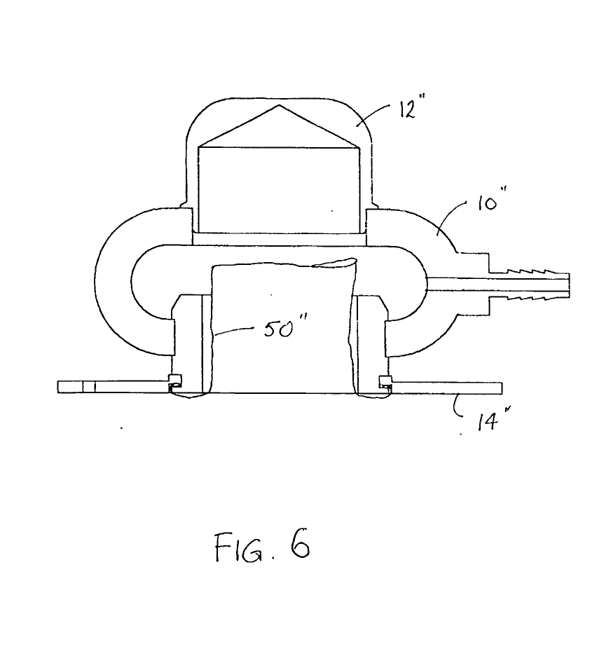

In the alternative form of the invention shown in FIGS. 5 and 6, an elastic sleeve 50″ again is passed under the bottom of the base 14″, which snaps into the housing 10″, gripping the rolled bottom of the sleeve. A cap 12″ is inserted into a hole at the top of the housing. The slots 60 shown in FIG. 5 are intended to receive elastic retaining straps which may be used to secure the device to the person. Such retainers may, of course, be used with the other embodiments.

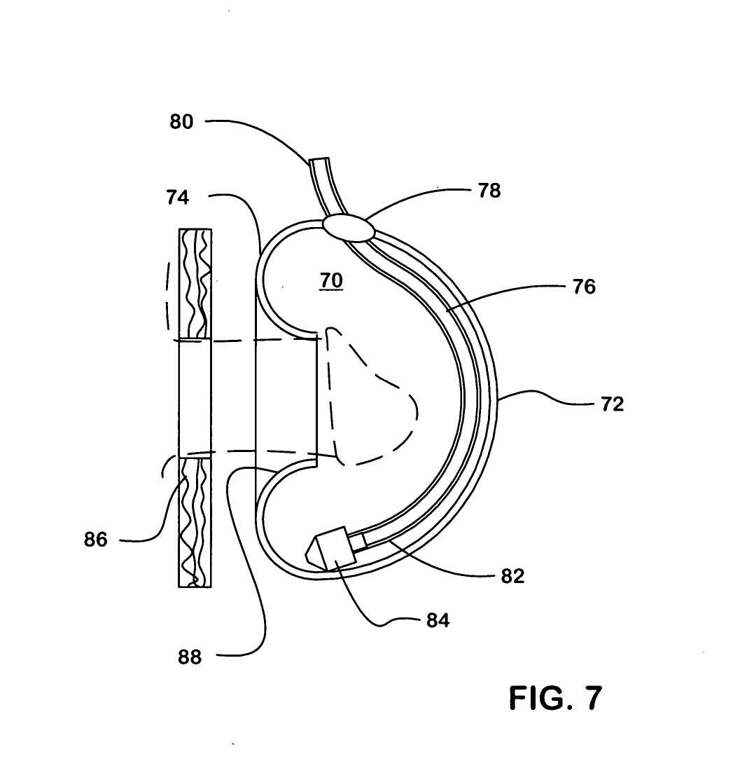

The catheter shown in FIG. 7 has a hollow body 70, the front 72 of which is generally hemispherical and the reverse 74 of which is generally hemitoroidal or half-donut shaped. This shape make urine spillage almost impossible, even when the wearer is lying on his back. It should be understood that the shapes of the front and rear of the body need not be exactly hemispherical and hemitoroidal, and that some variation from geometric exactness may be tolerated or even preferable.

The hollow body is constructed of a plastic polymer (preferably PET). A surgical rubber pickup tube 76 extends through a hole 78 at the periphery of the body, so that the proximal end 80 of the tube is outside the body, and the distal end 82 is within the body.

A clunk weight pickup 84 is installed on the distal end of the tube to keep it at the lowest point in the catheter, so that it collects urine no matter which side the patient sleeps on; this minimizes the amount of urine within it. The proximal end of the pickup tube is connectable to a vacuum chamber (not shown) where collected urine is retained to later be measured, examined, or disposed of. The vacuum is provided by a small vacuum pump (not shown), which draws air through the catheter, thus eliminating ammonia and helping avoid skin irritation and breakdown. The unit's design gives a user or patient an advantageous alternative to condom-type catheters or other invasive designs.

In use, a disc-shaped absorbent shield 86 is first placed over the penis, to catch any spillage which may occur due to loss of power, vacuum pump failure, or dislodging of the catheter. The shield keeps the catheter from sealing to the patient's body and applying vacuum directly to any tissues. The shield also makes the device more comfortable to wear. The penis is then inserted through the aperture 88 in the reverse surface into the body of the device. If the aperture is about the same size as, or slightly smaller than, the penis, no separate seal may be needed; however, it is possible that a flaccid sealing sleeve as described above, or other sealing device could be installed at the aperture.

The catheter preferably includes a vent to keep any negative pressure or vacuum from affecting any tissues. The volume of air drawn through the catheter is comparable to that used in a dentist's office or in a surgical operating room. The ball catheter is non-invasive, comfortable, and works remarkably well. The clunk weight preferably is bronze but could be made of a less expensive material coated, if necessary, with an inert layer to keep urine from reacting with it.

The invention could be further modified so as to be a self-contained portable unit, and custom versions may also be developed for use in road vehicles or aircraft.

Inasmuch as the invention is subject to modification and variation, it is intended that the foregoing description should be regarded as only illustrative of the invention defined by the claims that follow.

1. An external urinary catheter comprising
a hollow body sized to fit over the penis, said body having a closed front surface and a reverse surface having an aperture for receiving the penis,
a length of tubing extending through a wall of the body to collect urine from within the catheter body, and
a weight at the distal end of the tubing, within the hollow body, to keep the distal end of the tubing at the lowest point in the hollow body, regardless of the orientation of the hollow body.

2. The invention of claim 1, wherein said front surface is substantially hemispherical.

3. The invention of claim 2, wherein the reverse surface is substantially toroidal.

4. The invention of claim 1, wherein the reverse surface is substantially toroidal.

5. The invention of claim 1, further comprising means for connecting the vacuum tubing to a source of vacuum.

6. The invention of claim 1, further comprising a hermetic seal between the hollow body and the tubing where the tubing passes through the wall of the body.

7. The invention of claim 1, wherein the tubing is flexible.

8. The invention of claim 7, wherein the tubing is just long enough to permit the weight to contact the wall of the body, in any orientation thereof.

9. The invention of claim 7, wherein the shape of the body is such that the weight just contacts the wall of body, in any orientation thereof.

10. The invention of claim 1, wherein the weight is a body having a longitudinal axis, a through bore passing along said axis, and fitting at one end of the bore for receiving the distal end of the tubing.
